(19) United States
(12) Patent Application Publication
(10) Pub. No.: US 2024/0277631 A1
(43) Pub. Date: Aug. 22, 2024

(54) SMALL MOLECULE DRUG (ELP-004) TO PREVENT BONE LESIONS CAUSED BY MULTIPLE MYELOMA

(71) Applicants: West Virginia University Board of Governors on behalf of West Virginia University, Morgantown, WV (US); Temple University-Of the Commonwealth System of Higher Education, Philadelpha, PA (US); University of Pittsburgh-Of the Commonwealth System of Higher Education, Pittsburgh, PA (US)

(72) Inventors: John B. Barnett, Morgantown, WV (US); Werner Geldenhuys, Morgantown, WV (US); Jonathan Soboloff, Phildelphia, PA (US); Harry Colbert Blair, Pittsburgh, PA (US); Jamie McCall, Morgantown, WV (US)

(21) Appl. No.: 18/438,308

(22) Filed: Feb. 9, 2024

Related U.S. Application Data
(60) Provisional application No. 63/484,093, filed on Feb. 9, 2023.

(51) Int. Cl. A61K 31/136; A61K 31/69; A61P 35/00

(52) U.S. Cl. CPC A61K 31/136; A61K 31/69; A61P 35/00

(57) ABSTRACT

A method of inhibiting the development of osteoclasts in patients having multiple myeloma comprising administering to a patient having multiple myeloma a therapeutically effective amount of N-Methyl-dichloropropionaniline (i.e. also known as ELP-004) for inhibiting the development of osteoclasts. The method includes wherein said N-Methyl-dichloropropionaniline is administered to said patient in a pharmaceutically acceptable vehicle.

### CROSS-REFERENCE TO RELATED APPLICATIONS

This application claims benefit of U.S. Provisional Application No. 63/484,093 filed on Feb. 9, 2023, the disclosure of which is hereby incorporated by reference in its entirety.

### STATEMENT REGARDING FEDERALLY SPONSORED RESEARCH

This invention was made with government support under Grant No. AR074812 awarded by the National Institute of Health, and Grant No. BX002490-06A1 awarded by the Department of Veterans Affairs. The government has certain rights in the invention.

### BACKGROUND OF THE INVENTION

### Field

Embodiments relate to inhibition of development of osteoclasts in patients having multiple myeloma.

### Description of Related Art

Multiple myeloma is the second most frequent type of blood cancer, afflicting 32,270 adults each year within the United States and causing approximately 12,800 deaths. Survival rates have steadily increased over the last decade, with the 5-year survival exceeding about 54%. The significant improvements in survival and clinical outcomes over the years are primarily driven by new treatments, including novel small molecules, immunotherapies, autologous stem cell transplantation, and enhanced supportive care.

Although survival has improved, one of the most common complications with multiple myeloma patients is destruction of the patient's bone as the cancerous cells propagate within bone marrow. Multiple myeloma is a bone marrow cancer. The cancer cells of multiple myeloma overstimulate the production of osteoclasts, cells specific for bone resorption. Osteoclasts are cells that, in combination with osteoblasts, remodel bone constantly. In patients having multiple myeloma, the overtly stimulated osteoclasts cause severe bone lesions, as the bone is degraded without being replaced, which add to the problems with treating the myeloma cancer.

Few efficacious treatments are available to prevent bone loss. The only available drugs approved clinically to control bone erosion are biologicals (i.e. adalimumad and infliximab) that are being used for the treatment of arthritis. The mode-of-action of these biologicals is to primarily inhibit inflammatory response cytokines, e.g., TNF-alpha, and secondarily, inhibit osteoclast activity. The inhibition of the inflammatory response by these biologicals is their primary mode of action and is desirable for rheumatoid arthritis patients. However, these immunosuppressive properties also subject patients to the risk of infection, weakness, and gastrointestinal issues. Collateral side effects of biologicals on the non-inflammatory immune functions is deemed an acceptable side effect. However, up to 40% of rheumatoid arthritis patients cannot take these drugs because of these and other side effects.

Another option are bisphosphonates (e.g., alendronate, ibandronate, risedronate, and zoledronic acid) which have shown to be effective at slowing bone loss and/or hardening bone in some cases. However, this does not cure the condition and includes negative side effects such as bone, joint, or muscle pain along with nausea, difficulty swallowing, and gastric ulcers. As such, there is an unmet medical need for drugs that inhibit osteoclast-mediated bone loss in those with cancer.

### SUMMARY

Disclosed herein are methods and compositions for prevention and treatment of bone loss by inhibiting osteoclastogenesis. As disclosed herein, an ion-channel antagonist, ELP-004, suppressed osteoclast maturation and strongly suppressed the development of bone lesions in patients having multiple myeloma.

This disclosure provides a method of inhibiting the development of osteoclasts in patients having multiple myeloma comprising administering to a patient having multiple myeloma a therapeutically effective amount of N-Methyl-dichloropropionaniline (i.e. also known as ELP-004) for inhibiting the development of osteoclasts.

This disclosure provides a method of inhibiting multiple myeloma cell stimulation of the production of osteoclasts in patients having multiple myeloma comprising administering a therapeutically effective amount of N-Methyl-dichloropropionaniline (i.e. also known as ELP-004).

Also provided in this disclosure is a method of treating a patient having multiple myeloma comprising administering to the patient a therapeutically effective amount of N-Methyl-dichloropropionaniline (i.e. also known as ELP-004) in combination with a standard of care chemotherapeutic agent. The standard of care chemotherapeutic agent is, for example, Bortezomib. ELP-004 is known as N-Methyl-dichloropropionaniline, N-methyl-DCPA (i.e. N-MeDCPA), and N-(3,4-dichlorophenyl)-N-methyl propenamide.

Also disclosed herein is a method of inhibiting the development of osteoclasts in patients having multiple myeloma, comprising administering to a patient having multiple myeloma a therapeutically effective amount of N-Methyl-dichloropropionaniline (also known as ELP-004) for inhibiting the development of osteoclasts. In exemplary embodiments of this method, the N-methyl-dichloropropionaniline is administered in a pharmaceutically acceptable vehicle. In exemplary embodiments, the pharmaceutically acceptable vehicle is administered via oral administration.

Disclosed herein is a method of inhibiting multiple myeloma cell stimulation of the production of osteoclasts in patients having multiple myeloma comprising administering a therapeutically effective amount of N-methyl-dichloropropionaniline (also known as ELP-004) to said patient for inhibiting multiple myeloma cell stimulation of the production of osteoclasts. In exemplary embodiments, the N-methyl-dichloropropionaniline is administered to patients in a pharmaceutically acceptable vehicle. In exemplary embodiments, the pharmaceutically acceptable vehicle is administered via oral administration.

Disclosed herein is a method of treating a patient having multiple myeloma comprises administering to the patient a therapeutically effective amount of N-methyl-dichloropropionaniline (also known as ELP-004) in combination with a standard of care chemotherapeutic agent. In exemplary embodiments, the N-methyl-dichloropropionaniline can be administered to the patient in a pharmaceutically acceptable vehicle. In exemplary embodiments the pharmaceutically acceptable vehicle is administered to the patient via oral administration. In exemplary embodiments, the standard of care chemotherapeutic agent can be Bortezomib. In exemplary embodiments, the ELP-004 inhibits overstimulation of osteoclast production without interfering with standard of care chemotherapy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Other features and advantages of the compositions and processes disclosed herein will be apparent to those skilled in the art reading the following detailed description in conjugation with the exemplary embodiments described and/or illustrated in the drawings, wherein;

FIG. 1 shows a graphical representation of U266 cell counts after treatment with varying concentrations of N-MeDCPA at 24, 48, 72 and 96 hour time intervals along with a graphical representation of U266 cell concentration from apoptosis or necrosis after treatment with varying concentrations of N-MeDCPA. The U266 cells were incubated in the presence of the noted concentrations of ELP-004, and the viability of the cells was determined at the indicated times using the Axenin V method. The IC50 was calculated using two methods to be 153.7 μM.

FIG. 2A-F shows data representative of ELP-004 inhibition of basal calcium content and associated NFATcl nuclear translocation in cytokine-induced, but not RANKL-induced osteoclastogenesis. Murine BMSMCs were differentiated to form osteoclasts under varying conditions, where at each time point, cytosolic Ca²⁺ levels (A-C0 and NFAT nuclear translocation (D-F) were measured to show ELP-004 (100 nM) had no effect on NFAT translocation in RANKL-induced osteoclastogenesis, but significantly inhibited both TNFα— and LTB4-induced changes in both endpoints.

FIGS. 3A-C shows a representation of ELP-004 inhibition of matrix resorption activity of osteoclasts as differentiated by cytokines. Murine BMSMCs were differentiated to form osteoclasts on Osteo-Assay surface plates. The sizes of pits formed by the resorptive activity of osteoclasts differentiated under the three conditions were analyzed at days 7, 14, and 21. Average pit sizes of 10-30 pits are shown. No resorption was observed in the presence of ELP-004 irrespective of conditions although osteoclasts differentiated in the presence of cytokines developed earlier and exhibited greater activity, especially in the presence of TNFα.

FIGS. 4A-C shows data corresponding to ELP-004 having minimal effect on RANKL-induced osteoclastogenesis. Murine BMSMCs were grown for up to 21 days in media supplemented with 50 ng/ml of m-CSF and 100 ng/mL RANKL. Each experiment was repeated a minimum of three times. Cells were fixed and stained for Tartrate Resistant Acid Phosphatase (TRAP) activity at days 1, 7, 14, and 21. The number of trap positive osteoclast-like cells (out of 100 cells) were counted for each time point, along with TRAP intensity, as shown in schematic A. In schematic B, the number of nuclei for each cell counted and the average number of nuclei per cell for 100 cells is shown at each time point. In schematic C, mRNA expression of osteoclast markers such as TRAP, VATPASE A3, VATPASE D2, and Calcitonin Receptor (CALCR) were measured at days 7 and 14 for both control and cells treated by RT-PCR, all showing that ELP-004 has no significant effect on TRAP activity, multinucleation or the expression of osteoclast markers in cells differentiated into osteoclasts by RANKL (100 ng/ml).

FIGS. 5A-C shows ELP-004 blocking TNFα-induced Osteoclast Fusion. (A_C) Murine BMSMCs were grown for up to 21 days in media supplemented with 50 ng/ml m-CSF, 1 nh/mL RANKL and 2 ng/ml TNFα. Each experiment was repeated a minimum of three times. Cells were fixed and stained for Tartrate Resistant Acid Phosphatase (TRAP)_activity at days 1, 7, 14 and 21. In schematic A, the number of TRAP positive osteoclast-like cells (out of 100 cells) were counted for each time point; TRAP intensity was also calculated. In schematic B, at each time point the number of nuclei for each cell was counted and the average number of nuclei per cell for 100 cells is shown. In schematic C, mRNA expression of osteoclast markers such as TRAP5, VATPASE A3, VATPASE D2 and Calcitonin Receptor (CALCR) were measured at days 7 and 14 for both control cells and cells treated by RT-PCR to show ELP-004 treatment completed abrogated TRAP activity and severely attenuated multinucleation in cells differentiated into osteoclasts by RANKL/TNFα. The expression patterns of osteoclast markers was unaffected by ELP-004, indicating selective inhibition of specific features of osteoclastogenesis.

FIGS. 6A-C show ELP-004 blocking LTB4-induced Osteoclast Fusion. (A-C) Murine BMSMCs were grown for up to 21 days in media supplemented with 50 ng/ml m-CSF, 1 ng/ml RANKL and 50 nM LTB4. Each experiment was repeated a minimum of three times. Cells were fixed and stained for Tartrate Resistant Acid Phosphatase (TRAP) activity at days 1, 7, 14 and 21. In schematic A, the number of TRAP positive osteoclast-like cells (out of 100 cells) were counted for each time point; TRAP intensity was also calculated. In schematic B, at each time point, the number of nuclei for each cell was counted and the average number of nuclei per cell for 100 cells is shown. In schematic C, mRNA expression of osteoclast markers such as TRAP5, VATPASE A3, VATPASE D2 and Calcitonin Receptor (CALCR) were measured at days 7 and 14 for both control and treated cells by RT-PCR. ELP-004 treatment completed abrogated TRAP activity and severely attenuated multinucleation in cells differentiated into osteoclasts by RANKL/LTB4. The expression patterns of osteoclast markers was unaffected by ELP-004, indicating selective inhibition of specific features of osteoclastogenesis.

FIG. 7 shows bone loss evaluation in the animal model of multiple myeloma. Shown are cross-sections of distal femur micro-computer tomographic images from mice treated with 60 mg/kg ELP-004 or vehicle control at 72h intervals after the engraftment of human 2×10⁶ myeloma cells (U266 cells). Confirmation of engraftment was verified by measuring serum human IgE levels.

FIGS. 8A-G shows data obtained from daily oral treatment with ELP-004-LNE, as it reduces arthritis phenotypes in SKG mice. The SKG mice are arthritis prone due to a Zap70 mutation, with arthritis initiated by a zymosan A injection. (A) Schematic representation of experimental design. (B) Total body weight on d56 was unaffected by treatment. (C) The concentration of ELP-004 was measured in the bone marrow to assess concentrations at the site of target engagement. (D) Weekly AI assessments were analyzed using a repeated-measures two-way ANOVA with Sidak's correction for multiple comparisons. (E) The left hind paw of each mouse was assessed by microCT. Examples of bone erosion indicative of arthritis are indicated by white arrows. (F) Porosity and bone mineral density (BMD) of the tarsus (shown in white box) was quantified in each mouse. (G) Porosity and BMD were assessed in the calcaneus (white box). ELP-004 reduced porosity and increased bone mineral density in the tarsus and calcaneus of the female mice.

FIGS. 9A-D show data plots obtained to show that ELP-004-LNE improves bone strength parameters in the SKG mouse model of arthritis. Osteoporosis is one of the most common extra-articular complications of RA and is characterized by decreased BMD and increased risk of fractures. It is estimated to occur in about 27.6% of RA patients or approximately double the rate of the general population (Moshayedi et al, Scientific Reports, 2022). In the US, osteoporotic fractures account for about one-third of RA-related mortality (Fardellone et al, J. Clin. Med., 2020). In mouse models, trabecular microarchitecture in the long bones and spine is a measurement of bone strength. (A,B) Femurs and (C,D) L5 vertebrae in the spine were visualized using microCT. The trabecular bone analyzed in the region of interest is outlined in white. (B) ELP-004-treated female mouse femurs had increased bone strength with treatment based on trabecular number, bone surface/tissue volume (BS/TV), bone volume/tissue volume (BV/TV), and BMD as compared to vehicle-treated controls. (D) ELP-004-treated mice from both sexes exhibited increased L5 vertebral bone strength with treatment.

FIG. 10 shows a graphical representation of the elimination of ELP-004 after i.v. administration. ELP-004 was reconstituted in 20% hydroxypropyl-beta-cyclodextrin (HPbCD) and administered to female C57B16 mice (3/timepoint/route of administration) in the fed state. Following i.v. (2 mg/kg) was isolated from whole blood at 5, 15, and 30 min and at 1, 2, 4, 7, and 24 h. ELP-004 concentration was measured by LC-MS/MS. The data was fit to a two-compartment model and slopes for the a and B elimination were determined.

### DESCRIPTION

As disclosed herein, the methods and novel compounds, or any pharmaceutically acceptable salts thereof, of this disclosure provide for treatment of bone lesions in patients. The bone lesions can result from certain types of cancer, autoimmune disease, or other metabolic disorders. Specifically, as used herein, the methods and compounds are used for treatment of patients with multiple myeloma.

The disclosure provides a method of inhibiting the development of osteoclasts in patients having multiple myeloma comprising administering to a patient having multiple myeloma a therapeutically effective amount of N-Methyl-dichloropropionaniline (i.e. also known as ELP-004) for inhibiting the development of osteoclasts.

As used herein, the term “patient” means members of the animal kingdom, including but not limited to, human beings.

As used herein, the term “effective amount” or “therapeutically effective amount” refers to that amount of any of the present compounds, salts thereof, and/or compositions required to bring about a desired effect in a patient. The desired effect will vary depending upon the illness or disease state being treated. For example, the desired effect may be inhibiting osteoclastogenesis, lessening bone lesions, and/or preventing overstimulation of osteoclast production, any of which may be the desired therapeutic response. On its most basic level, a therapeutically effective amount is that amount of a substance needed to inhibit osteoclastogenesis. The term “inhibits or inhibiting” as used herein means reducing growth/replication.

As will be understood by one skilled in the art, a therapeutically effective amount of said compound can be administered by any means known in the art, including but not limited to, injection, parenterally, intravenously, intraperitoneally, orally or, where appropriate, topically.

As used herein, the term “ELP-004” is defined as N-Methyl-dichloropropionaniline. Other names include N-methyl-DCPA (i.e. N-MeDCPA), and N-(3,4-dichlorophenyl)-N-methyl propenamide. N-MeDCPA (i.e. ELP-004; i.e. N-(3,4-dichlorophenyl)-N-methylpropanamide) is a haloanilide compound of Formula I:

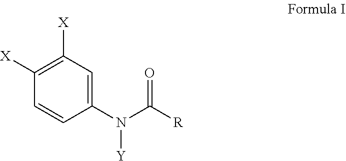

wherein each X is chlorine, Y is a methyl group, and R is an ethyl group. N-MeDCPA is synthesized as described in U.S. Pat. No. 10,682,320, which is incorporated by reference herein. ELP-004 (N-MeDCPA) precludes metabolizing to two toxic metabolites, N—OH-dichloroaniline and 6-OH-dichloroaniline. In vitro assays for toxicity caused by ELP-004 using Jurkat cells show 50% toxicity at 800 mM and <2% toxicity at 200 mM. The Contract Research Organization Illinois Institute of Toxicology Research Institute (IITRI) performed 1× escalating toxicity in rats and determined that the oral LD₅₀ was >2.5 g/kg body weight.

Excellent in vivo efficacy to prevent bone erosion associated with arthritis using two mouse models, was achieved using 150 mM intraperitoneally in an N-MeDCPA nanoparticle preparation. Thus, ELP-004 has high potential for use as a drug to prevent or reduce bone erosion.

In exemplary embodiments, a method is provided of inhibiting multiple myeloma cell stimulation of the production of osteoclasts in patients having multiple myeloma comprising administering a therapeutically effective amount of N-Methyl-dichloropropionaniline (i.e. also known as ELP-004).

In exemplary embodiments, administration ELP-004 to said patient for inhibition of development of osteoclasts in patients having multiple myeloma is via a pharmaceutically acceptable vehicle.

As used herein, the term “pharmaceutically acceptable vehicle” refers to any pharmaceutically acceptable carrier known in the art, absent compatibility problems with the compounds of the invention. Generally, pharmaceutically acceptable vehicles include for example, but not limited to, physiologic saline and 5% dextrose in water.

ELP-004 is not water soluble. Those persons of ordinary skill in the art will understand that ELP-004 may be solubilized in ethanol and then diluted in sterile saline. These methods include wherein the route of administration may be, for example but not limited to, the intravenous, intramuscular, or subcutaneous routes, or via the oral route of administration.

In exemplary embodiments, the pharmaceutically acceptable vehicle is administered to said patient via oral route of administration.

Another aspect of this disclosure provides a method of treating a patient having multiple myeloma comprising administering to the patient a therapeutically effective amount of N-Methyl-dichloropropionaniline (i.e. also known as ELP-004) in combination with a standard of care chemotherapeutic agent. An example of a standard of care chemotherapeutic agent is Bortezomib (Velcade®, Takeda Pharmaceuticals U.S.A., Inc). ELP-004 prevents overstimulation of osteoclast production without interfering with standard of care chemotherapy.

As used herein, a “standard of care chemotherapeutic agent” refers to any treatment that is accepted by medical experts as a proper treatment for a certain type of disease, which here is cancer. An example of a standard of care chemotherapeutic agent is Bortezomib (Velcade®, Takeda Pharmaceuticals U.S.A., Inc.). As disclosed herein, a selection of or a combination therapeutics approved for multiple myeloma or other plasma cell neoplasms may be administered in combination with ELP-004. These therapeutics may include, but are not limited to, Idecabtagene Vicleucal, Mephalan Hydrochloride, Mephalan, Pamidronate Disodium, Carmustine, Bortezomib, Carfilzomib, Carmustine, Ciltacabtagene Autoleucel, Cyclophosphamide, Daratumumab, Doxorubicin Hydrochloride Liposome, Elotuzumab, Elranatamab-bcmm, Idecabtagene Vicleucel, Isatuximab-irfc, Ixazomib Citrate, Lenalidomide, Plerixafor, Pomalidomide, Isatuximab-irfc, Selinexor, Talquetamab-tgvs, Teclistamab-cqyv, Thalidomide, and/or Zoledronic Acid.

In exemplary embodiments, the ELP-004 in combination with the standard of care chemotherapeutic agent is administered to the patient in a pharmaceutically acceptable vehicle.

In exemplary embodiments, the pharmaceutically acceptable vehicle is administered to the patient via oral administration.

In exemplary embodiments, the standard of care chemotherapeutic administered in combination with ELP-004 is Bortezomib.

In exemplary embodiments, the ELP-004 in combination with the standard of care chemotherapeutic inhibits overstimulation of osteoclast production without interfering with standard of care chemotherapy.

As disclosed herein, ELP-004 directly inhibits osteoclastogenesis, and thus has a different mode of action over biologicals (i.e. adalimumad and infliximab). ELP-004 can be combined with standard of care chemotherapeutic agents for treatment of multiple myeloma patients. Because ELP-004 inhibits osteoclastogenesis directly, ELP-004 does not interfere or exacerbate chemotherapy activity nor does it affect immune function at therapeutic dose levels. ELP-004 inhibits the development of osteoclasts in a dose dependent manner.

ELP-004 has been evaluated for its ability to cross the blood brain barrier. Studies have found that ELP-004 does not cross the blood brain barrier. Further, ELP-004 does not cause problems with dopamine transport. Other medications, such as but not limited to bupropion, are known to cause problems due to dopamine transporter binding.

ELP-004 has a half-life when administered orally that indicates that it is essentially eliminated within 4 hours. The short half-life and the relative time to Cmax indicates dopaminergic side effects would occur within 1-2 hours of administration of ELP-004. ELP-004 has no deleterious effect on induction of CYP enzymes.

It is well within the skill of one practicing in the art to determine what dosage, and the frequency of this dosage, that will constitute a therapeutically effective amount for each individual patient, depending on the severity or progression of cancer or cancer cells and/or the type of cancer, as well as the severity or progression of bone lesions. It is also within the skill of one practicing in the art to select the most appropriate method of administering the compounds based upon the needs of each patient.

### Examples

Initial treatment with N-methyl dichloropropionaniline (N-MeDCPA, ELP-004) was examined. The N-MeDCPA was designed and synthesized and tested in laboratory experiments. A treatment scheme with plated cells was devised using N-MeDCPA as compared to a DMSO vehicle only, along with 100 μM and 200 μM dosages on U266 cell samples. U266 is a B lymphocyte isolated from the peripheral blood of a 53-year-old, male patient with myeloma and is commonly used in immune system disorder research and immunology research. The cells were allowed to incubate then counted under a 10× lens.

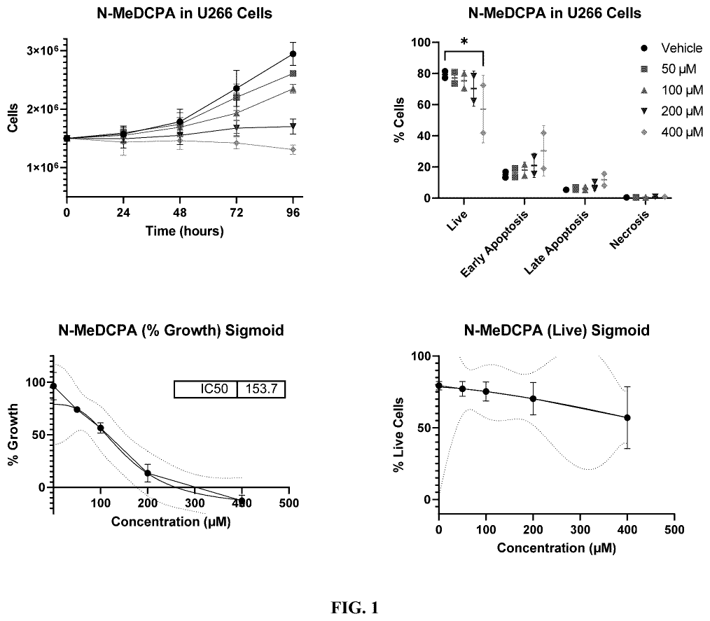

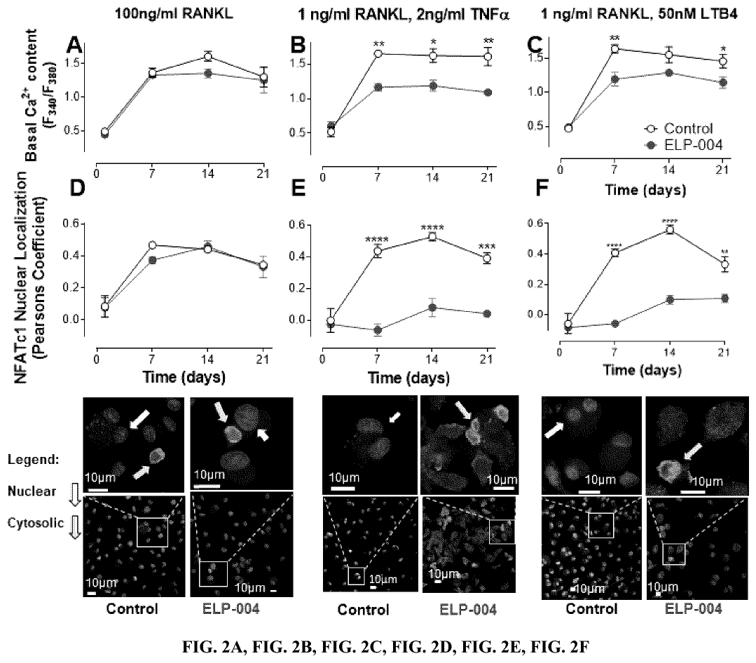

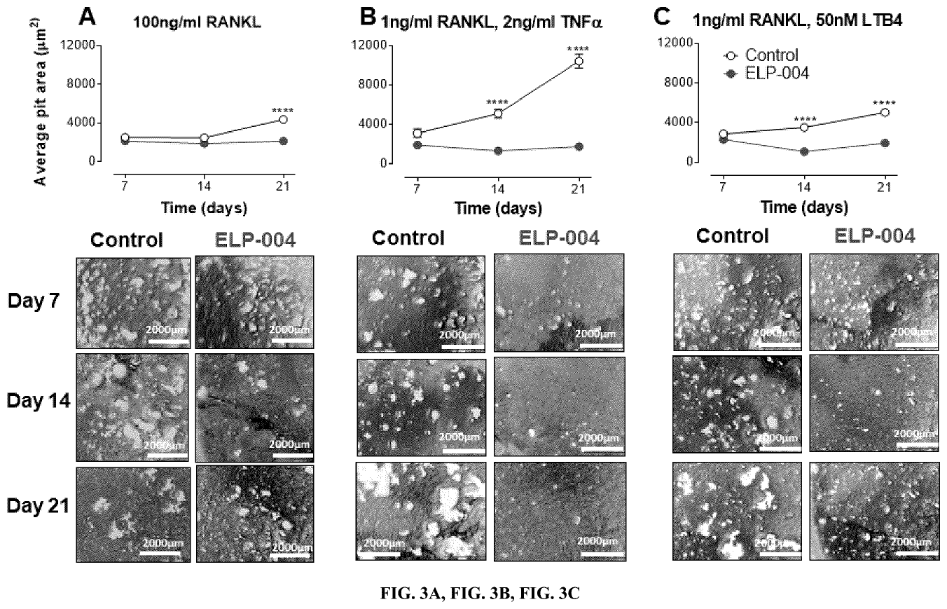

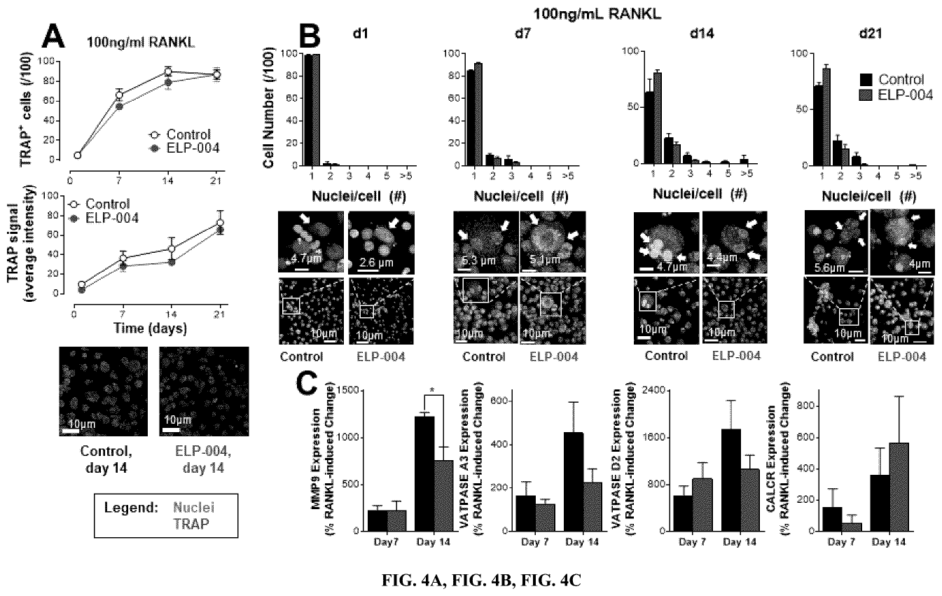

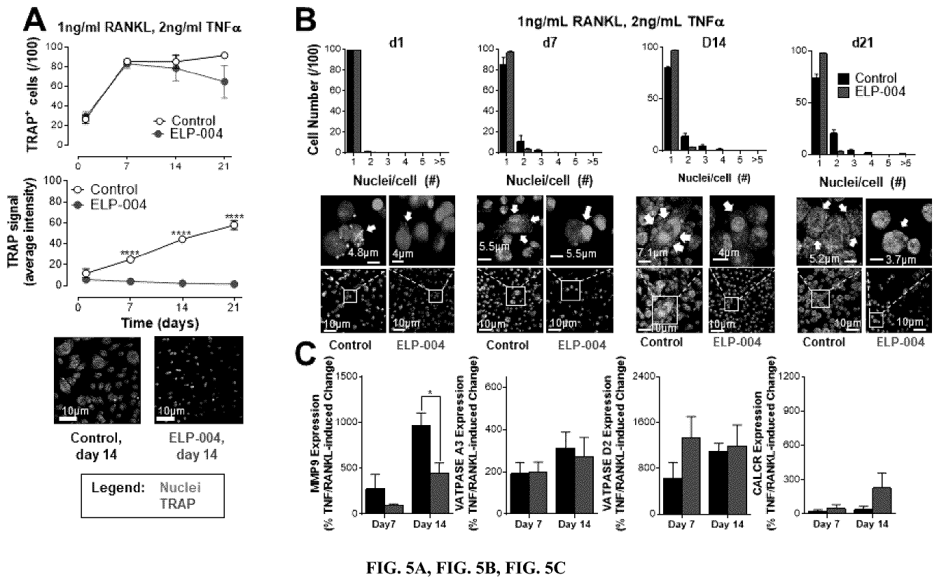

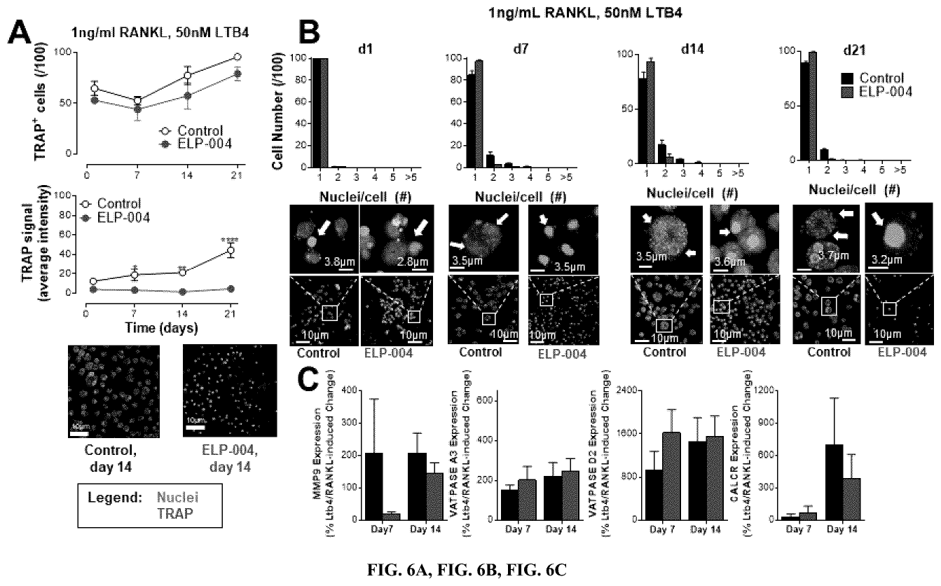

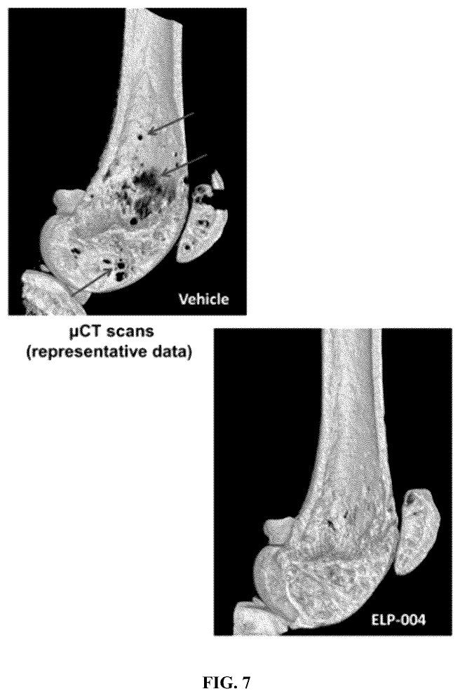

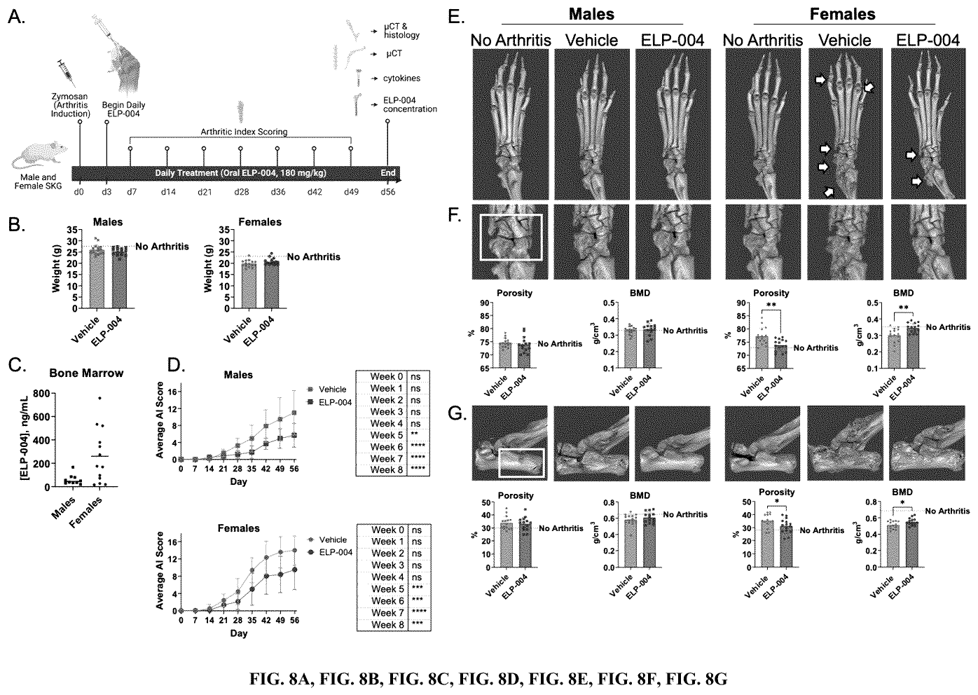

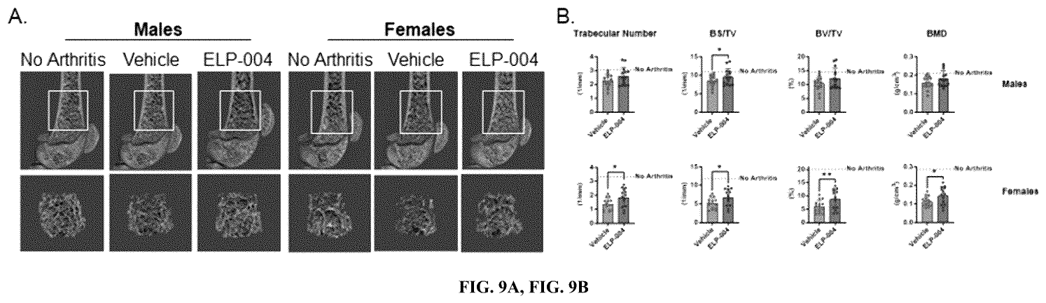

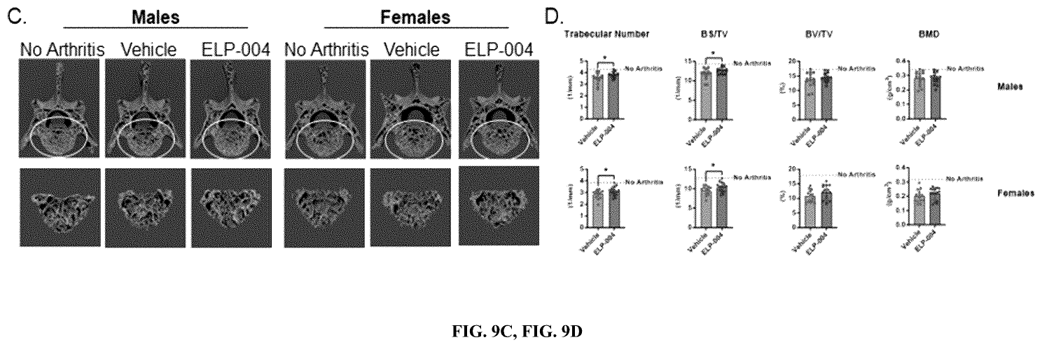

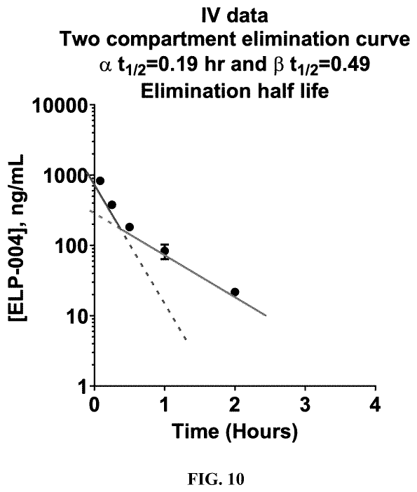

As may be seen in FIG. 1, the growth curve of cell samples as treated with the varying concentrations versus a DMSO vehicle can be observed. The number of cells per milliliter was calculated after examination and counting under the lens. The results show, in intervals of 48 to 96 hours, the gradual growth of the DMSO vehicle is accelerated while the growth of the treated cells is inhibited.

### Results

### Development and Safety Profile of N-MeDCPA as a Lead Compound

ELP-004 (i.e. N-MeDCPA) was specifically developed to preclude metabolizing to two toxic metabolites, N—OH-dichloroaniline and 6-OH-dichloroaniline. In vitro assays for toxicity caused by ELP-004 using Jurkat cells show 50% toxicity at 800 mM and <2% toxicity at 200 mM. The Contract Research Organization Illinois Institute of Toxicology Research Institute (IITRI) performed 1× escalating toxicity in rats and determined that the oral LD₅₀ was >2.5 g/kg body weight.

Thus, ELP-004 is useful as a drug to prevent or reduce bone lesions in multiple myeloma patients. ELP-004 (N-MeDCPA) was synthesized as described in U.S. patent Ser. No. 10/682,320.

The effect of ELP-004 (i.e. N-MeDCPA) on U66 cell (a myeloma cell line) proliferation is described above. These experiments demonstrate that ELP-004 does not stimulate the growth or proliferation of myeloma cells. These experiments show that ELP-004 inhibits myeloma cell proliferation, and while this inhibition of myeloma cells does not approach an effective level of efficacy to be considered a chemotherapeutic agent, it does indicate that ELP-004 would not protect a myeloma tumor from the effects of a standard of care chemotherapeutic agent. Those persons of ordinary skill in the art recognize that if ELP-004 stimulated a myeloma tumor, this would have indicated that this treatment would not be clinically viable.

In exemplary embodiments, the N-MeDCPA is administered with Bortezomib to reach a target of approximately 30% cell apoptosis.

### IND-Enabling Studies to Evaluate Pharmacokinetics and Pharmacodynamic Properties

Depicted in table 1 is pharmacokinetic data of ELP-004 administered via i.v, p.o., and s.c. ELP-004 was reconstituted in 20% hydroxypropyl-beta-cyclodextrin (HPbCD) and administered to female C57B16 mice (3/timepoint/route of administration) in the fed state. Following i.v. (2 mg/kg), s.c. (2 mg/kg), or p.o. (10 mg/kg) administration, plasma was isolated from whole blood at 5, 15, and 30 min and at 1, 2, 4, 7, and 24 h. Plasma from p.o. was not isolated at 5 min. ELP-004 concentration was measured by LC-MS/MS (see below). The limit of quantification was 10 ng/ml and samples below that were not used in the analyses. PK parameters were calculated using Phoenix WinNonlin (Certara, NJ, USA).

TABLE 1

| Route of Administration | i.v. | p.o. | s.c. |
|---|---|---|---|
| Dose | 2 mg/kg | 10 mg/kg | 2 mg/kg |
| t_{1/2} (h) | 0.493 | 1.19 | 0.445 |
| tₘₐₓ (h) | — | 0.25 | 0.25 |
| C₀ (ng/mL) | 1220 | — | — |
| Cₘₐₓ (ng/mL) | — | 211 ± 42.9 | 461 ± 39.0 |
| AUCₗₐₛₜ (h*ng/mL) | 374 ± 23.2 | 227 ± 18.0 | 307 ± 31.7 |
| AUC_{Inf} (h*ng/ml) | 390 | 245 | 322 |
| AUC Extr (%) | 3.96 | 7.29 | 4.55 |
| V_{z} (L/kg) | 3.65 | — | — |
| Vₛₛ (L/kg) | 2.48 | — | — |
| CL (mL/min/kg) | 85.5 | — | — |
| C₀ (μM) | 5.26 | — | — |
| Cₘₐₓ (μM) | — | 0.909 ± 0.185 | 1.986 ± 0.100 |
| F (%) | — | 12.6 | 82.6 |

Depicted in table 2 is cytochrome P450 phenotyping and inhibition by ELP-004. The stability of ELP-004 was tested in the presence of recombinant enzymes CYP1A2, CYP2B6, CYP2C8, CYP2C9, CYP2C19, CYP2D6, and CYP3A4. ELP-004 (1 μM) was incubated with recombinant enzymes and NADPH. The amount of ELP-004 remaining at each timepoint was analyzed by LC-MS/MS. Incubations in control recombinant enzymes were also performed to assess non-enzymatic mediated stability. The intrinsic clearance of ELP-004 was determined. ELP-004 was not metabolized by CYP2C9 and CYP2D6 recombinant enzymes. The ability of ELP-004 to reversibly inhibit the CYP isoforms CYP1A2, CYP2B6, CYP2C8, CYP2C9, CYP2C19, CYP2D6, and CYP3A4 was assessed in human liver microsomes. A decrease in the formation of the metabolite compared to vehicle control is used to calculate an IC₅₀ value. Metabolite peak area ratio response as a percentage of vehicle control is plotted against the test compound concentration. ELP-004 did not inhibit any CYP over the concentration range assessed (0.02-25 μM) and therefore no IC₅₀ values were calculated.

TABLE 2

| Isoform | CYP1A2 | CYP2B6 | CYP2C8 | CYP2C9 | CYP2C19 | CYP2D6 | CYP3A4 |
|---|---|---|---|---|---|---|---|
| ELP-004 t_{1/2} | 9.73 | 8.51 | 127 | >180 | 99.2 | >180 | 143 |
| (min) | | | | | | | |
| Recombinant | | | | | | | |
| CYP CL_{INT} | 2.85 | 0.815 | 0.219 | <0.154 | 0.279 | <0.154 | 0.193 |
| (uL/min/mg) | | | | | | | |
| Inhibition IC₅₀ | >25 | >25 | >25 | >25 | >25 | >25 | >25 ^{‡} |
| (μM) | | | | | | | |

CL_{INT} = Intrinsic Clearance,
^{‡} = midazolam and testosterone

It will be appreciated by those persons skilled in the art that changes could be made to embodiments of the present invention described herein without departing from the broad inventive concept thereof. It is understood, therefore, that this invention is not limited by any particular embodiments disclosed but is intended to cover the modifications that are within the spirit and scope of the invention, as defined by the appended claims.

The invention claimed is:

1. A method of inhibiting the development of osteoclasts in patients having multiple myeloma comprising administering to a patient having multiple myeloma a therapeutically effective amount of N-Methyl-dichloropropionaniline.

2. The method of claim 1 including wherein said N-Methyl-dichloropropionaniline is administered to said patient in a pharmaceutically acceptable vehicle.

3. The method of claim 2 including wherein said pharmaceutically acceptable vehicle is administered to said patient via the oral route of administration.

4. A method of inhibiting multiple myeloma cells stimulation of the production of osteoclasts in patients having multiple myeloma comprising administering a therapeutically effective amount of N-Methyl-dichloropropionaniline to said patient for inhibiting multiple myeloma cells stimulation of the production of osteoclasts.

5. The method of claim 4 including wherein said N-Methyl-dichloropropionaniline is administered to said patient in a pharmaceutically acceptable vehicle.

6. The method of claim 5 including wherein said pharmaceutically acceptable vehicle is administered to said patient via the oral route of administration.

7. A method of treating a patient having multiple myeloma comprising administering to the patient a therapeutically effective amount of N-Methyl-dichloropropionaniline in combination with a standard of care chemotherapeutic agent for treating said patient.

8. The method of claim 7 including wherein said N-Methyl-dichloropropionaniline is administered to said patient in a pharmaceutically acceptable vehicle.

9. The method of claim 8 including wherein said pharmaceutically acceptable vehicle is administered to said patient via the oral route of administration.

10. The method of claim 7 including wherein said standard of care chemotherapeutic agent is Bortezomib.

11. The method of claim 7 including wherein said N-Methyl-dichloropropionaniline inhibits overstimulation of osteoclast production without interfering with standard of care chemotherapy.
